(19) United States
(12) Patent Application Publication
(10) Pub. No.: US 2023/0346883 A1
(43) Pub. Date: Nov. 2, 2023

(54) COMPOSITIONS FOR AND METHODS OF TREATMENT WITH MUTANT A1 ADENOSINE RECEPTOR PLASMID cDNAs

(71) Applicant: Endacea, Inc., Research Triangle Park, NC (US)

(72) Inventor: Constance N. Wilson, Raleigh, NC (US)

(73) Assignee: Endacea, Inc., Research Triangle Park, NC (US)

(21) Appl. No.: 17/734,574

(22) Filed: May 2, 2022

(51) Int. Cl. A61K 38/17; A61K 39/395; A61K 9/51; A61P 35/00; A61K 45/06; A61K 38/21; C12N 15/85

(52) U.S. Cl. CPC A61K 38/177; A61K 39/39558; A61K 9/5123; A61P 35/00; A61K 45/06; A61K 38/217; C12N 15/85; C12N 2800/107

(57) ABSTRACT

Novel mutant A1 adenosine receptor cDNA sequences and plasmids. Novel mutant A1 adenosine receptor cDNA sequences and plasmids which can improve immune cells ability to kill cancer cells.

A text file having the name ENDA016_Sequences_2022-05-02_ST25.txt with the date of creation being May 2, 2022, byte size 23.0 KB (23,621 bytes), of synthetic protein sequences is incorporated herein and is included herein in their entirety by reference. No new matter has been added. Applicant respectfully requests the “Sequence Listing” previously submitted as a PDF file be removed.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to novel mutant A1 adenosine receptor sequences and plasmids which can improve immune cells ability to kill cancer cells.

### Description of Related Art

The A1 adenosine receptor is a member of the G-protein coupled receptor family and consists of a single polypeptide chain that spans the membrane seven times, with the amino terminal being extracellular and the carboxy terminal being intracellular.

### Tumor Evasion by Cancer Cells and Immune Cells

A number of cells play a role in the containment of cancer, including tumor infiltrating lymphocytes, regulatory T cells, CD8⁺ cytotoxic T cells, CD4⁺ helper T cells, myeloid-derived suppressor cells, immune inhibitory B cells, tumor associated macrophages, natural killer cells, dendritic cells, and antigen presenting cells (APCs). Tumor cells may reprogram myeloid cells to create an immunosuppressive tumor environment and drive tumor progression. For example, factors associated with tumor immunosuppression include tumor growth factor-β and interleukin (IL)-10, as well as ligands that down modulate tumor infiltrating lymphocyte activity, such as programmed death ligand (PD-L1). As such, an approach to the treatment of cancer is based on immune checkpoint blockade therapy, such as antibodies that block cytotoxic T lymphocyte antigen 4 (CTLA-4) and PD-1, chimeric antigen receptor (CAR) T cell immunotherapy, and therapeutic vaccines, such as T cell and dendritic cell vaccines with or without tumor antigens or tumor neoantigens.

### Cancer Immunotherapies

Tumors evade destruction by the immune system by a number of different mechanisms, including multiple regulatory pathways and cells.

Based on the recognition of the role of the immune system in controlling tumorigenesis and tumor progression, a number of cancer immunotherapeutic agents, including check point inhibitors, targeting immune tolerance and combinations thereof have been or are currently being investigated. “Most cancer immunotherapeutic agents are categorized as 1) drugs that target tumor immune evasion via blockade of negative regulatory signals (e.g., co-inhibitory checkpoints and tolerogenic enzymes) and 2) drugs that directly stimulate immunogenic pathways (e.g., agonists or co-stimulatory receptors), or a combination of co-inhibitory and co-stimulatory agents. Other immunostimulatory strategies include enhancers of antigen presentation (e.g., dendritic and T cell vaccines), the use of exogenous recombinant cytokines, oncolytic viruses, and cell therapies using native or modified antigen-competent immune cells”.

Drugs that Target Immune Tolerance Via Checkpoint Inhibitors

Co-inhibitory agents include, but are not limited to, antibodies against CTLA-4 and PD-1, antagonist of T cell immunoglobulin and mucin domain (TIM-3), antagonist of v-domain immunoglobulin-containing suppressor of T cell activation (VISTA, also known as PD-1 homolog), antagonist of lymphocyte activation gene 3 (LAG-3), drugs that target T cell immunoglobulin (TGIT), TGFβ receptor type 1 inhibitor, and CD19 and CD3 bispecific antibodies. Other strategies that target checkpoint inhibitors include adaptive resistance to checkpoint inhibition.

Drugs that Enhance Antitumor Responses, Co-Stimulatory Agents

Co-stimulatory agents include, but are not limited to, cancer vaccines, either whole cell or specific peptide antigen preparations, e.g., tumor specific antigens or tumor associated antigens. Antigen presenting cells, such as macrophages or dendritic cells, display these tumor antigens to activate B and T cells. In general, tumor vaccines are administered with an adjuvant nonspecific immune stimulant. For example, granulocyte macrophage colony stimulating factor (GM-CSF) may be administered with a dendritic vaccine to increase the immune response to the tumor. Vaccines, including dendritic cell vaccines may be treated with a tumor specific antigen. Tumor specific antigens are highly tumor specific and expressed only on tumor cells. Tumor associated antigens are more widely expressed in both tumor and nontumor cells. Most vaccine peptide targets are restricted to specific HLA haplotypes, however, and may include somatic mutations of cancer antigens (i.e., tumor neoantigens). Molecules, such as heat shock proteins may serve as a chaperone for tumor peptides.

Antigen-presenting cells may be obtained from any source, such as peripheral blood mononuclear cells, peripheral blood monocytes, circulating stem cells, stem cells or precursor cells derived from bone marrow, peripheral blood, cord blood; or antigen-presenting cells may be found in tissue parenchyma, generated in vitro, obtained from a commercial source or cloned. Antigen-presenting cells include, but are not limited to, monocytes, macrophages, dendritic cells, Langerhans cells, lymphocytes, hematopoetic stem cells, peripheral blood stem cells, peripheral blood mononuclear cells, B cells, veiled cells, interdigitating and follicular cells, splenocytes, thymocytes, microglia, Kupffer cells, endothelial cells, fibroblasts, eosinophils, and any cell displaying HLA-peptide complexes on its cell surface(U.S. Pat. No. 8,247,231). Agents capable of increasing the number of A1 adenosine receptors on the antigen-presenting cell plasma membrane include, but are not limited to, cisplatin, dexamethasone, daunorubicin, doxorubicin, mitoxantrone, carbamazepine, adenosine receptor antagonists, nucleotide sequences encoding the A1 adenosine receptor, for example, cDNA encoding the human A1 adenosine receptor, allosteric enhancers, such as PD 81,723, which increases the affinity and binding of an A1 adenosine receptor ligand for A1 adenosine receptors and coupling of the receptor to the G protein; contacting the cells with divalent cations, including magnesium and calcium; and/or contacting the cells with adenosine deaminase, or immunomodulators or priming agents, such as lymphokines, MDP, MTP, MTP-PE, IFN-γ, PMA, GM-CSF, fMLP, or FLT3 ligand, and protein kinase inhibitors. The number of A1 adenosine receptors may also be increased by subjecting the cells to ischemic conditions.

Drugs that stimulate an antitumor response include checkpoint stimulators, including CD 28, tumor necrosis factor receptor family including glucocorticoid-induced tumor necrosis factor receptor (GITR), OX40, and 4-1BB (CD137), CD40, stimulator of interferon genes (STING) agonists, and cytokines, including interferons (IFNs), including INF-α and INF-β, toll receptor agonists, IL-2, IL-15, IL-17, IL-21, and IL-7. Strategies that produce antitumor responses include oncolytic virus therapies, adoptive T cell therapy, wherein T cells are artificially enriched with tumor specific antigens, CAR T cell therapies, T cell receptor (TCR) therapies, and DNA-based, RNA-based and mRNA-based vaccines.

It has previously been reported that activation of the A1 adenosine receptor produces a tumoricidal effect in vitro. In vivo, however, due to the immunosuppression in the microenvironment of tumors and a specific defect in the A1 adenosine receptor protein on immune cells, activation of A1 adenosine receptors on human immune cells may fail to kill tumor cells and contain the growth of a cancer.

### BRIEF SUMMARY OF THE INVENTION

The present invention relates to compositions and methods of treatment using mutant A1 adenosine receptor plasmid cDNAs, one of which significantly reduces tumor volume in a mouse model of cancer in vivo.

Accordingly, in one embodiment, there is a composition for mutant amino acid sequences for the cDNA for the A1 adenosine receptor for inclusion in a plasmid selected from the group comprising:
a) an amino acid sequence as set forth in FIG. 1 (SeqID no. 1);
b) an amino acid sequence as set forth in FIG. 2 (SeqID no. 2);
c) an amino acid sequence as set forth in FIG. 3 (SeqID no. 3);
d) an amino acid sequence as set forth in FIG. 4 (SeqID no. 4);
e) an amino acid sequence as set forth in FIG. 5 (SeqID no. 5);
f) an amino acid sequence as set forth in FIG. 6 (SeqID no. 6);
g) an amino acid sequence as set forth in FIG. 7 (SeqID no. 7); and
h) an amino acid sequence as set forth in FIG. 8 (SeqID no. 8).

Accordingly, in another embodiment, there is a method of inhibiting the proliferation of a cancer cell and reducing the size of a tumor comprising multiple cancer cells, the method comprising:
a) administering a plasmid directly to the cancer cell, tumor, or an organ in which the tumor is expressed, a mutant A1 adenosine receptor plasmid cDNA wherein the plasmid delivers one or more mutant A1 adenosine receptors selected from the group consisting of:
   i. an amino acid sequence as set forth in FIG. 1 (SeqID no. 1);
   ii. an amino acid sequence as set forth in FIG. 2 (SeqID no. 2);
   iii. an amino acid sequence as set forth in FIG. 3 (SeqID no. 3);
   iv. an amino acid sequence as set forth in FIG. 4 (SeqID no. 4);
   v. an amino acid sequence as set forth in FIG. 5 (SeqID no. 5);
   vi. an amino acid sequence as set forth in FIG. 6 (SeqID no. 6);
   vii. an amino acid sequence as set forth in FIG. 7 (SeqID no. 7); and
   viii. an amino acid sequence as set forth in FIG. 8 (SeqID no. 8).

### BRIEF DESCRIPTION OF THE DRAWINGS

FIGS. 1 through 8 are the mutant amino acid sequences for Sequence ID (SeqID) numbers 1 through 8.

FIG. 9 is an exemplary plasmid utilizing a mutant amino acid sequence of FIGS. 1 through 8.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

Combination Treatments with A1 Adenosine Receptor Mutant Plasmid cDNA

The treatment with a mutant A1 adenosine receptor plasmid cDNA of the present invention may be administered with other co-inhibitory or co-stimulatory cancer immunotherapy agents described above. The treatment with a mutant A1 adenosine receptor plasmid cDNA may be administered with both co-inhibitory and co-stimulatory cancer immunotherapy agents described above. The treatment with a mutant A1 adenosine receptor plasmid cDNA may be administered with surgery, radiation, chemotherapeutics and immunotherapies, such as toll receptor agonists and oncolytic viruses.

Moreover, treatment with a mutant A1 adenosine receptor plasmid cDNA may be administered with A1 adenosine receptor agonists, priming agents, protein kinase inhibitors, endotoxin, A2a adenosine receptor antagonists, agents which may increase A1 adenosine receptor expression, tyrosine phosphatase inhibitors, adenosine deaminase, allosteric enhancers, chemotherapeutic agents, radiation treatment, surgery, immunotherapies, vaccines including dendritic cell, T cell, B cell, natural killer cell, and stem cell vaccines, and tumor specific associated antigens, or neoantigens in combination with the plasmid cDNA for the mutant A1 adenosine receptor.

A1 adenosine receptor agonists include, but are not limited to, adenosine; cyclohexyladenosine; various N6-substituted A1 adenosine receptor agonists including, but not limited to N6 cyclopentyladenosine, N6 R-phenylisopropyladenosine, 2-chloro N6 cyclopentyl adenosine (CCPA), N6 (p-sulfophenyl) alkyl and N6 sulfoalkyl derivatives of adenosine (such as N6-(p-sulfophenyl) adenosine); 1-deaza analogues of adenosine including, but not limited to N6 cyclopentyl 1-2-chloro-1-deaza adenosine (1-deaza-2-CI-CPA); N6 cycloaklyladenosines; N6 bicycloalkyladenosines; ribose modified adenosine receptor analogues including, but not limited to 3′-deoxy-R-PIA.

The binding of these A1 adenosine receptor agonists to A1 adenosine receptors and their activation may be enhanced by an allosteric enhancer such as P(2-amino-4,5-dimethyl 1-3-thienyl)-[3-trifluoromethyl phenyl] methadone. Additional A1 adenosine receptor agonists are known in the art. Optimal dosing and administration schedules may be determined using routine methods known to those in the art.

The cells may be treated or “primed” to enhance A1 adenosine receptor activity. Cells may be primed prior to activation. For example, antigen-presenting cells may be primed using any priming agent known in the art including, but not limited to, PMA; lipopolysaccharide (LPS); platelet activating factor (PAF); tumor necrosis factor alpha (TNFα) or thrombin; f-met-leu-phe (FMLP); zymosan; macrophage stimulating factors including granulocyte macrophage colony stimulating factor (GM-CSF); ionomycin (for example in 1 μM amounts); calcium ionophore (such as A 23187, for example in 0.1-10 μM amounts); gamma interferon (IFNγ, for example in 1-150 units/ml amounts); supernatants of tumor cells; or bacterial products from gram positive organisms. Preferred priming conditions for the type of cell to be activated may be determined using routine methods known to those in the art.

Agents which may increase A1 adenosine receptor expression on immune cells include cisplatin, dexamethasone, daunorubicin, doxorubicin, mitoxantrone, and carbamazepine. Conditions such as ischemia and reperfusion or ischemia alone may be used to increase A1 AR expression on immune cells.

A2a adenosine antagonists include, but are not limited to, triazoloquinazoline (CGS15943); pyrazolo[4,3-e]-1,2,4-triazolo [1,5-C] pyrimidine derivatives such as 7-2(phenylethyl)-5-amino-2-(2-furyl)-pyrazolo-[4.3-e]-1,2,4 triazolo [1,5-c] pyrimidine; 8-(3-chlorostyryl)caffeine; 8-(3-isothiocyanatostyryl)caffeine; E-1,3-diakyl-7-methyl-8-(3,4,5-trimethoxy-styryl)xanthines, (E)-1,3-dipropyl-7-methyl-8-(3,4-dimethoxystyryl)xanthine; 4-(2-[7-amino-2-{2-furyl}{1,2,4}triazolo{2,3-a}{1,3,5}triazin-5-yl-amino] ethyl)phenol; 7-deaza-9phenyladenines.

As used herein, the administration of two or more compounds “in combination” means that the two compounds are administered closely enough in time that the presence of one alters the biological effects of the other. The two compounds may be administered simultaneously (i.e., concurrently) or sequentially. Additionally, simultaneous administration may be carried out by mixing the compounds prior to administration, or by administering the compounds at the same point in time but at different anatomic sites or using different routes of administration.

The phrases “concurrent administration”, “administration in combination”, “simultaneous administration”, or “administered simultaneously” as used herein interchangeably means that the compounds are administered at the same point in time or immediately following one another. In the latter case, the two compounds are administered at times sufficiently close that the results observed are indistinguishable from those achieved when the compounds are administered at the same point in time.

Moreover, methods described herein can be performed “in combination” or “simultaneously” with other methods described herein or with known methods or treatments of interest, including surgery, radiation, chemotherapeutics, androgen deprivation therapy, and immunotherapies to achieve the desired result. Immunotherapies may include toll receptor agonists and oncolytic viruses. Other treatments of interest include ablative therapies, i.e., high-intensity focused ultrasound (HIFU), interstitial laser ablation therapy (ILAT), vascular targeted photodynamic therapy (PDT), and cryotherapy (CT).

Delivery Methods for Mutant A1 Adenosine Receptor Plasmid cDNA

Delivery methods for the mutant A1 adenosine receptor cDNA include direct injection into the tumor, intravenous, inhalational, subcutaneous, rectal, and topical delivery of the plasmid cDNA for the mutant A1 adenosine receptor. Moreover, the mutant A1 adenosine receptor cDNA may be delivered into an organ tissue where the tumor is expressed. The topical delivery of the mutant plasmid cDNA may include the use of a patch with or without microneedles. Topical or intratumor administration of the plasmid cDNA for the mutant A1 AR may include the use of endoscopy, surgery, ultrasound, interventional radiology, MRI, CT scan, and PET imaging to locate the tumor.

Novel delivery platforms for cancer immunotherapies that are in development include nanoparticles, implants, scaffolds including biomaterial implant scaffolds and injectable biomaterial scaffolds, biomaterials, cell-based platforms, controlled release systems, and transdermal delivery. Materials including lipids, polymers, e.g., poly (lactic-co-glycolic acid (PLGA)), cationic protamines, peptides, e.g., placental growth factor 2 (PLGF2), matrix metalloproteinases, adhesion ligands, small interfering RNA (siRNA), polyethylene glycol, and metals have been used to develop these delivery technologies. Moreover, injectable hydrogels and microneedles may be used to develop delivery systems for cancer immunotherapies. Antibody fragments may be conjugated to the surface of nanoparticles via thiolmaleimide click chemistry. Viral delivery systems may include lentiviruses, adeno-associated viruses, and the Sendai virus which are capable of delivering mRNA.

Non-viral vectors may be engineered to deliver mRNA to dendritic cells. These non-viral vectors prevent degradation of the mRNA by serum endonucleases. They also avoid renal clearance and prevent nonspecific interactions. Once the mRNA is taken up by the dendritic cells, it is processed into smaller peptide epitopes that bind to major histocompatibility complex (MHC) class I or class II molecules. MHCs are trafficked to the cell surface of which then bind to CD8⁺ (cytotoxic) T cells or CD4⁺ (helper) T cells, respectively, leading to cytotoxic T cell response or antigen-specific antibody response, respectively. These technologies may be engineered to deliver cDNAs and plasmid cDNAs.

Liposome-based drug delivery systems with vascular adhesion receptor E-selectin alone or with immune cytokines (e.g., TRAIL), have been developed to bind immune cells in the circulation. These immune cells may kill tumor cells in the tumor and circulating tumor cells.

Lipid-based nanoparticles have been shown to be efficacious in targeting dendritic cells without the need for antibodies or adhesion ligands. They typically consist of an ionizable lipid, a helper lipid, phospholipid, cholesterol, and polyethylene glycol (PEG)-lipid, such as 1,2-diastearoyl-sn-glycero-3-phosphoethanolamine-N-[maleimide(PEG)-2000] (DSPE-PEG2000-maleimide). Lipids, including cationic lipids, 1,2-DI-O-octa-decenyl-3-trimethylammonium-propane (DOTMA), 1,2-dioleoyl-3-trimethylammonium-propane (DOTAP), 1,2-distearoly-sn-glycero-3-phophocholine (DSPC), sufosuccinimidyl 4-(N-maleimidomethyl) cyclohexane-1-carboxylate (SMCC), and zwitterionic lipid 1,2-dioleoyl-sn-glycero-3-phosphoethanolamine (DOPE) have been used in the development of delivery systems for cancer immunotherapies. To overcome the side effects of cationic lipids, ionizable lipid-like materials have been designed. Polymeric systems, e.g., dendrimers, have also been developed.

Subunit vaccines containing purified peptides, proteins, polysaccharides in combination with molecular adjuvants have been developed to boost the immune system. Moreover, amphiphilic vaccine conjugates consisting of an antigen or adjuvant conjugated to albumin-binding lipophilic tail and linked to a functionalized PEG block have been developed to improve the immune response to subunit vaccines in draining lymph nodes. Subunit vaccines in combination with a tumor antigen-targeting antibody, an engineered version of IL-2, and an anti-PD-1 mAb, recruit numerous innate and adaptive immune cells.

Matrix-binding molecular conjugates have been designed to avoid immune side effects associated with systemic administration of checkpoint inhibitors. Moreover, angiotensin inhibitors, e.g., losartan, may reduce extracellular matrix stiffening in the cancer microenvironment of solid tumors, prime the tumor microenvironment to cancer immunotherapies, and stimulate the immune system.

Externally or internally triggered drug delivery systems includes methods, e.g., those that are triggered mechanically, by pH, light, near-infrared light, or ultrasound, and the use of materials and modalities (e.g., microbubbles and ultrasound).

The use of cells such as somatic cells, CD34 and other hematopoietic stem cells may be used to deliver the mutant plasmid cDNA to the tumor or an organ in which the tumor is expressed.

Direct intratumor administration or administration to an organ tissue that expresses a tumor may include the use of electroporation, adenoviruses, transfection with plasmid vectors containing mutant cDNA encoding A1 adenosine receptors and other methods well known in the art.

### Biomaterials for Localized Immunotherapy

To minimize adverse systemic effects and lower the dose of cancer immunomodulatory agents, delivery systems have been designed for local and sustained release. Mineral oils and polymeric microspheres are used for local delivery. Specifically, Montanide ISA 52, a commercially available mixture of light mineral oils, has been used in immunotherapy clinical trials. A biodegradable polymer, such as poly(D,L-lactic-co-hydroxymethyl glycolic acid (PLHMGA) or PLGA with or without microparticles has been used for the slow release of immunomodulatory agents.

The injection of mesoporous silica rods into tissue can provide a 3D structure into which immune cells, e.g., dendritic cells, can be recruited and interact with other immune cells.

Microneedle-based platforms loaded with immunotherapeutic nanocarriers have been developed for delivery of cancer immunotherapeutic agents. Nanoparticle encapsulation of cancer immunotherapeutic agents may include conjugation to hyaluronan. Following delivery, the nanoparticle is dissociated by hyaluronidase resulting in local release of the cancer immunomodulatory agent into the tumor environment.

Another example of an implantable biomaterial, porous poly(lactide-co-glycolide) (PLG) scaffolds were encapsulated with GM-CSF to stimulate dendritic cell recruitment and proliferation. Encapsulation of GM-CSF, CpG oligodeoxynucleotides (CpG-ODNs), CC-chemokine ligand 20 (CCL20), or FMS-related tyrosine kinase 3 ligand (FLT3L) in combination with tumor cell lysates, specific tumor antigens, or synthetic neoantigens may be immobilized on immunomodulatory scaffolds to enhance the recruitment of dendritic cells, which traffic out of the scaffold to lymph nodes where they stimulate antitumor immunity.

Scaffold-based delivery systems designed with the use of materials, such as alginate hydrogels, gelatin, and mesoporous silica microrods, can be administered by injection and create immunogenic microenvironments for the activation and recruitment of immune cells. For example, mesoporous silica microrods coated with a fluid lipid bilayer, anti-CD3 and anti-CD28 antibodies, and IL-2 to create an APC-mimetic scaffold for T cell expansion. Nanoparticles have been treated with cationic polymer polyethyleneimine (PEI) to stimulate proinflammatory cytokine production that may increase the immunogenicity of tumor neoantigens.

An alternative to silica, biodegradable hydrogels have been designed for the local delivery of cancer immunotherapies with and without a chemotherapy. One example of a hydrogel includes injectable poly(vinyl alcohol) (PVA), which is responsive to reactive oxygen species present in high levels in the tumor microenvironment. Following injection into the tumor environment, the hydrogel is degraded and releases chemotherapeutic therapies and immunomodulatory agents to kill cancer cells and stimulate antitumor immunity, respectively.

Many systems including nanoparticles, scaffolds, hydrogels, and cells can be loaded with multiple therapeutic agents chosen on the basis of targets identified in patient cancer biopsy samples, thus providing a personalized therapeutic approach to the treatment of cancer.

Transdermal delivery systems have been developed to deliver local sustained release of immunomodulatory anticancer agents to avoid adverse systemic effects and to minimize the required dose of the immunomodulatory agent. These delivery systems include a degradable microneedle patch which can penetrate the immune-rich epidermis. Microneedles typically consist of a biodegradable polymer, hyaluronic acid, and are loaded with pH-sensitive nanoparticles containing immunotherapeutic anticancer agents. Microdelivery systems may include immune-modulating agents, e.g., 1-methyl-DL-tryptophan (I-MT), an inhibitor of the immunosuppressive enzyme, indoleamine 2,3-dioxygenase (IDO), and melanin, with and without tumor cell lysates, tumor specific antigens, and tumor neoantigens. When exposed to near-infrared light, the melanin generates heat which causes the local release of inflammatory cytokines, adjuvants, and other antigens which attract and activate immune cells, e.g., dendritic cells in the endogenous tissue.

T cell therapy delivery technologies include surface conjugated nanoparticles, in situ T cell engineering via DNA nanocarriers, biomaterial-based implants for local adoptive T cell delivery, and the use of synthetic artificial antigen-presenting cells. T cell engineering with delivery systems consisting of adjuvant-loaded nanoparticles that are chemically conjugated to the surface of donor T cells, scaffolds, or a combination may improve adoptive T cell therapies. Nanoparticles conjugated to the surface of T cells may improve the delivery of cancer immunotherapies and chemotherapeutics. The use of polymeric platforms may achieve long-term release of adjuvants and increase T cell proliferation in vivo.

To overcome the problems associated with adoptive T cell therapy, e.g., elaborate procedures and high costs, approaches to in situ engineering of T cells are in development. Nanocarriers with DNA are fabricated with poly(β-amino ester) (PBAE) for the encapsulation of the DNA into nanoparticles and functionalized with an anti-CD3 antibody to facilitate binding to T cells in the blood stream. Moreover, peptides containing microtubule-associated sequences and nuclear localization signals may facilitate the import of DNA and CAR genes into the nucleus of the T cell in situ. Furthermore, engineering T cells to express CARs with CRISPR-Cas9 for multiplexed genome editing may improve T cell delivery to organs with cancer.

Microfluidics-based technologies have been developed to deliver nucleic acids and macromolecules to immune cells ex vivo, including T cells, B cells, dendritic cells, macrophages, and other antigen presenting cells.

Polymeric scaffolds have been investigated for local adoptive T cell therapy directly to the tumor microenvironment. Polymeric scaffolds may be conjugated with T cell adhesion receptors, adjuvants, and immunomodulatory antitumor agents. Polymerized alginate scaffolds functionalized with a collagen-mimetic peptide are designed to deliver T cells and silica microtubules to enhance the proliferation of T cells in tumors.

Synthetic artificial antigen-presenting cells (aAPCs) may be functionalized with MHC class I immunoglobulin dimer and the co-stimulatory anti-CD28 signal required for T cell activation. Furthermore, aAPCs may be delivered with immunomodulating antitumor agents.

Moreover, macrophages may be engineered to directly attack tumor cells by inhibiting SHP substrate 1 (SIRPα), which prevents macrophages from attacking CD47-expressing tumor cells.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “proliferation” refers to cancer cells, e.g., in a tumor wherein the cells in the tumor increase in number.

As used herein, the term “reduce the size of a tumor” refers to killing at least a portion of cancer cells in a tumor.

As used herein, the term “organ” is a differentiated structure within the body (such as a heart, lungs, etc.) consisting of cells and tissues which perform some function within the body. It refers herein to an organ that expresses or contains a tumor.

As used herein, the term “plasmid” refers to a DNA structure possible to insert exogenous DNA, specifically a mutant A1 adenosine receptor plasmid cDNA, and is capable of replicating in a recipient cell.

As used herein, the term “administering” refers to the actual physical introduction of a plasmid of the invention into or onto (as appropriate) a cancer cell or cancerous tumor. Any and all methods of introducing the composition into the host or cell are contemplated according to the invention; the method is not dependent on any particular means of introduction and is not to be so construed. Means of introduction are well-known to those skilled in the art, and also are exemplified herein.

As used herein, administration “in combination” refers to both simultaneous and sequential administration of two or more agents or compositions. “Concurrent” or “combined administration”, as used herein, means that two or more agents or compositions are administered to a subject either (a) simultaneously, or (b) at different times during the course of a common treatment schedule. In the latter case, the two or more agents or compositions are administered sufficiently close in time to achieve the intended effect.

As used herein, the term “cancer” refers to a condition in a subject characterized by unregulated cell growth, wherein cancerous cells are capable of local invasion and/or metastasis to non-adjacent sites. As used herein, cancer cell“, cancerous cell”, or “tumor cell” refers to a cell characterized by this disordered cell growth and invasive properties.

The term “cancer, cancer cell, or cancer tumor” includes, without limitation, cancers of the heart system: sarcomas (angiosarcomas, fibrosarcomas, rhabdomyosarcomas, liposarcomas), myxoma, rhabdomyosarcomas, fibromas, lipomas, and teratomas; Lung cancer: bronchiogenic carcinoma (squamous cell, undifferentiated small cell, undifferentiated large cell, adenocarcinoma), alveolar (bronchiole) cancer, bronchial adenoma, sarcoma, lymphoma, chondromatomas hamartoma; Gastrointestinal cancer: esophagus (squamous cell carcinoma, adenocarcinoma, leiomyosarcoma, lymphoma), stomach (carcinoma, lymphoma, leiomyosarcoma), pancreas (pancreatic ductal adenocarcinoma, insulinoma, glucagonoma), Gastrinoma, carcinoid tumor, bipoma, small intestine (adenocarcinoma, lymphoma, carcinoid tumor, Kaposi's sarcoma, leiomyoma, hemangioma, lipoma, neurofibroma, fibroma), large intestine (adenocarcinoma, Thyroid adenoma, chorionic adenoma, hamartoma, leiomyoma); urogenital tract kidney cancer (adenocarcinoma, Wilms tumor [nephroblastoma], lymphoma, leukemia), bladder and urethra (squamous cell carcinoma, transitional cell carcinoma, gland) Cancer, prostate (adenocarcinoma, sarcoma), testis (seminoma, teratomas, embryonal carcinoma, teratocarcinoma, choriocarcinoma, sarcoma, stromal cell carcinoma, fibroma, fibroadenoma, adenoid tumor, lipoma); liver Cancer: liver cancer (hepatocellular carcinoma), cholangiocarcinoma, hepatoblastoma, hemangiosarcoma, hepatocellular adenoma, hemangioma; bone cancer: osteogenic sarcoma (osteosarcoma), fibrosarcoma, malignant fibrous histiocytoma, Chondrosarcoma, Ewing sarcoma, malignant lymphoma (reticular cell sarcoma), multiple myeloma, malignant giant cell tumor chordoma, osteochondroma (osteochondroma), benign chondroma, chondroblastoma, Cartilage myxofibromas, osteoid osteomas, and giant cell tumors; Cancer of the nervous system: Lid (osteomas, hemangiomas; granulomas, xanthomas, osteoarthritis), meninges (meningiomas, meningosarcoma, glioma), brain (astrocytoma, medulloblastoma, nerves) Glioma, ependymoma, germinoma [pineoblastoma], glioblastoma multiforme, oligodendroglioma, Schwann celloma, retinoblastoma, congenital tumor, spinal neurofibromas, meningiomas; Gynecological cancer: uterus (endometrial cancer), cervix (cervical cancer, preneoplastic cervical dysplasia), ovary (ovarian cancer [serous cystadenocarcinoma, mucinous cyst] Adenocarcinoma, unclassified carcinoma), granulosa-capsular cell tumor, Sertoli-Leydig cell tumor, anaplastic germoma, malignant teratoma, vulva (squamous cell carcinoma; carcinoma in situ, adenocarcinoma, fibrosarcoma, Melanoma), vagina clear cell carcinoma, squamous cell carcinoma, grape sarcoma (embryonic rhabdomyosarcoma), fallopian tube (carcinoma); blood system cancer: blood (myeloid leukemia [acute and chronic], Lymphoblastic leukemia, chronic lymphocytic leukemia, myeloproliferative disease, multiple myeloma, myelodysplastic syndrome), Hodgkin's disease, non-Hodgkin's lymphoma [malignant lymphoma], anaplastic large cell lymphoma (ALCL); skin cancer: malignant melanoma; basal cell carcinoma, squamous cell carcinoma, Kaposi's sarcoma, mole dysplastic nevi, lipoma, hemangioma, Merkel cell carcinoma; and adrenal cancer: including neuroblastoma, all forms of cancer, including but not limited to, forms of carcinoma, melanoma, sarcoma, lymphoma, and leukemia.

### EXPERIMENTAL

### Example 1: Synthesis of the Mouse Mutant A1 Adenosine Receptor (ADORA1) Plasmid cDNA

The synthetic gene mouse Protein X (ADORA1) was assembled from synthetic oligonucleotides and/or PCR products. The fragment was inserted into pMARQ (AmpR). The plasmid DNA was purified from transformed bacteria and concentration determined by UV spectroscopy. The final construct was verified by sequencing. The sequence identity within the insertion sites was 100%.

### Example 2: Formulation of Plasmid DNA (pDNA) for Mouse Mutant A1 Adenosine Receptor—Lipid Nanoparticle (LNP)

Lipids were blended in solvent and dried to a film. Lipid film was then placed under vacuum overnight to remove residual solvent. Lipid film was dissolved in ethanol to produce the organic phase. Plasmid DNA was received in phosphate-buffered saline. IFNγ was dissolved in phosphate-buffered saline and combined with plasmid DNA to make up the aqueous phase. The aqueous and organic phases were combined at controlled flow rates via microfluidics to produce lipid nanoparticles. Lipid nanoparticles were concentrated via tangential flow filtration, and then diafiltered fifteen times against phosphate-buffered saline. CD11c antibody was conjugated to lipid nanoparticles by incubating at 2° C. for approximately 18 hours. Conjugated lipid nanoparticles were then concentrated using centrifugal filtration. The resultant product was packaged under nitrogen into amber vials.

### Example 3: Treatment of PC3 Tumors (Human Prostate Carcinoma) in Athymic Nude Mice with a Mutant Mouse Plasmid cDNA for the A1 Adenosine Receptor Protein

### Materials and Methods

1. Animals:
   46 Athymic nude mice (ENVIGO, male, 5-6 weeks old, R #: 4269, PO #: 236172) were used in this study. All mice were ear tagged for identification purposes.
2. Animal Husbandry:
   Upon arrival, animals were examined to ensure that they were healthy. The animals were housed in autoclaved solid floor polycarbonate cages. Housing and sanitation were performed. All animal handling was performed in a laminar flow hood. Animals were housed in filter-topped cages within a HEPA filtered clean room.
3. Cell Culture and Implantation:
   PC3 cell line was obtained from American Type Culture Collection (Manassas, VA). The cells were cultured in 75 cm² flask containing Eagle's Minimum Essential Medium supplemented with 10% fetal bovine calf serum (FBS) and incubated at 37° C. in humidified atmosphere of 5% CO₂. As cells became 80% confluent, cultures were expanded to 150 cm² flasks, and expanded further until sufficient cells were available for injection.
   Cancer cells were subcutaneously injected into right flank, 10 million cells/each mouse for PC3 cells.
4. Assignment to Treatment Groups:
   The animals were ear tagged for identification purposes. The animals were assigned to 4 study groups with 10 mice in each group based upon tumor volume.
5. Treatment Regimen:
   Dosing started as shown in Table 1. The mice were dosed twice a week for 6 weeks.

TABLE 1
Treatment schedule

| | Number | | | Route of | |
|---|---|---|---|---|---|
| Group | of mice | Test Materials | Dose | dosing | Frequency |
| 1 | 10 | Vehicle (LNP2) | 50 μl | intratumor | Twice/week |
| 2 | 10 | Paclitaxel | 15 mg/kg | IV | Twice/week |
| 3 | 10 | cDNA-plasmid (LNP1) | 50 μg/50 μl | Intratumor | Twice/week |
| 4 | 10 | cDNA-plasmid-INFγ (LNP3) | 60 μg + 275 | intratumor | Twice/week |
| | | | ng/50 μl | | |

The Paclitaxel was stored at ambient temperature. Lipid nanoparticle (LNP) suspensions were stored at 4° C. Unopened vials were maintained in their original container at 4° C. until ready for use. 50 μl of LNP treatment was drawn via large bore needle (16G needle) from vial. All LNP treatments were allowed to reach room temperature by standing at ambient temperature. There was no heating. The large bore needle was replaced with a small-bore needle (25G) for tumor injection.
6. Tumor Monitoring:
   The xenograft tumors were measured three times a week with a digital caliper.
   Tumor volumes were calculated using the formula:

Tumor Volume=length×width×width×½
7. Statistical Analysis:
   Tumor sizes and body weights were analyzed using Student's t-test. P values <0.05 were considered as statistically significant.

### Results

1. There is no statistically significant difference in body weight between group 1 (Vehicle group) and group 3 (mutant cDNA-plasmid), as well as group 4 (mutant cDNA-plasmid-INFγ).
2. There is a statistically significant difference in tumor sizes between group 1 (Vehicle group) and group 3 (mutant cDNA-plasmid), as well as group 4 (mutant cDNA-plasmid-INFγ).
3. There is a statistically significant difference in tumor sizes between group 2 (Positive control group) and group 3 (mutant cDNA-plasmid), as well as group 4 (mutant cDNA-plasmid-INFγ).
4. Comparing to the treatment with Vehicle group (group 1), the treatment with mutant cDNA-plasmid-INFγ (group 4) resulted in 66% reduction in tumor volumes at DAY 45 and the treatment with mutant cDNA-plasmid (group 3) resulted in 53% reduction in tumor volumes at DAY 45. This reduction is indicative of significant treatment effects according to NIC criteria for tumor growth inhibition in xenograft model of human cancers.

### Effect of Treatment on Average Tumor Volume (Mm³):

| | | Therapy days | | | | | | | | | |
|---|---|---|---|---|---|---|---|---|---|---|---|
| Groups | Statistic | 0 | 3 | 5 | 7 | 10 | 12 | 14 | 17 | 19 | 21 |
| Group 1 | Mean | 114.5 | 183.0 | 193.6 | 215.6 | 259.8 | 316.7 | 341.5 | 382.2 | 430.6 | 455.3 |
| LNP2 | SD | 24 | 34 | 63 | 79 | 93 | 119 | 165 | 187 | 194 | 203 |
| Group 2 | Mean | 108.0 | 105.7 | 111.7 | 129.6 | 108.4 | 98.1 | 84.2 | 66.6 | 61.8 | 56.6 |
| Paclitaxel | SD | 26 | 24 | 26 | 26 | 25 | 28 | 29 | 32 | 27 | 31 |
| | p vs Grp 1 | 0.604 | 0.000 | 0.013 | 0.028 | 0.005 | 0.003 | 0.006 | 0.004 | 0.002 | 0.002 |
| Group 3 | Mean | 108.1 | 121 | 146.2 | 176.2 | 173.6 | 202 | 200.6 | 188.5 | 236.2 | 282.1 |
| LNP1 | SD | 26 | 28 | 26 | 26 | 30 | 5 | 65 | 50 | 77 | 122 |
| | p vs Grp 1 | 0.607 | 0.002 | 0.101 | 0.245 | 0.05 | 0.045 | 0.067 | 0.034 | 0.038 | 0.074 |
| | p vs Grp 2 | 0.993 | 0.207 | 0.008 | 0.001 | 0.000 | 0.000 | 0.000 | 0.000 | 0.000 | 0.000 |
| Group 4 | Mean | 107.8 | 120.1 | 156.6 | 167.4 | 186.3 | 197.6 | 182.7 | 176.5 | 228.3 | 232.3 |
| LNP3 | SD | 26 | 32 | 42 | 38 | 56 | 48 | 68 | 67 | 62 | 58 |
| | p vs Grp 1 | 0.592 | 0.002 | 0.208 | 0.173 | 0.096 | 0.039 | 0.045 | 0.027 | 0.033 | 0.027 |
| | p vs Grp 2 | 0.989 | 0.277 | 0.011 | 0.019 | 0.002 | 0.000 | 0.001 | 0.000 | 0.000 | 0.000 |

| | | Therapy days | | | | | | | | |
|---|---|---|---|---|---|---|---|---|---|---|
| Groups | Statistic | 24 | 26 | 28 | 31 | 35 | 38 | 40 | 42 | 45 |
| Group 1 | Mean | 528.4 | 560.7 | 570.6 | 691.3 | 893.8 | 968.0 | 1036.5 | 1197.8 | 1294.1 |
| LNP2 | SD | 252 | 254 | 242 | 312 | 340 | 392 | 518 | 774 | 820 |
| Group 2 | Mean | 48.7 | 34.6 | 31.1 | 26.6 | 25.3 | 24.4 | 24.2 | 27.2 | 17.8 |
| Paclitaxel | SD | 32 | 7 | 8 | 5 | 5 | 5 | 6 | 7 | 8 |
| | p vs Grp 1 | 0.002 | 0.002 | 0.001 | 0.001 | 0.001 | 0.001 | 0.002 | 0.007 | 0.006 |
| Group 3 | Mean | 264.6 | 294.3 | 319.7 | 408.3 | 505 | 555.5 | 605.4 | 628.1 | 604.6 |
| LNP1 | SD | 113 | 95 | 89 | 167 | 169 | 197 | 213 | 248 | 347 |
| | p vs Grp 1 | 0.033 | 0.032 | 0.034 | 0.058 | 0.023 | 0.033 | 0.073 | 0.103 | 0.072 |
| | p vs Grp 2 | 0.000 | 0.000 | 0.000 | 0.000 | 0.000 | 0.000 | 0.000 | 0.000 | 0.000 |
| Group 4 | Mean | 204.7 | 256.5 | 262.2 | 302.6 | 350.6 | 388.9 | 424.6 | 460 | 435.4 |
| LNP3 | SD | 71 | 104 | 125 | 151 | 141 | 166 | 175 | 196 | 275 |
| | p vs Grp 1 | 0.014 | 0.01 | 0.014 | 0.016 | 0.005 | 0.007 | 0.020 | 0.045 | 0.032 |
| | p vs Grp 2 | 0.000 | 0.000 | 0.000 | 0.000 | 0.000 | 0.000 | 0.000 | 0.000 | 0.001 |

Significance (p) was calculated by student's t-test.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

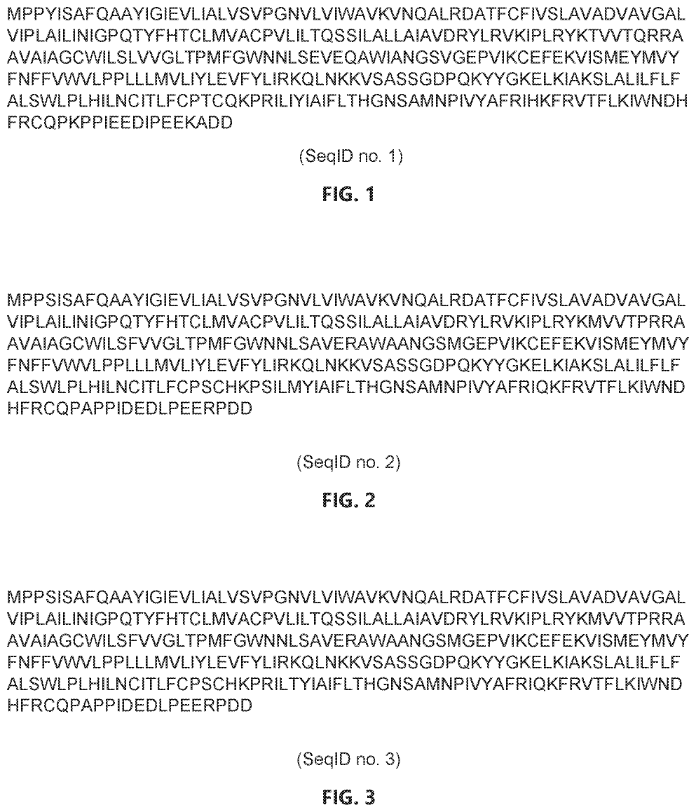

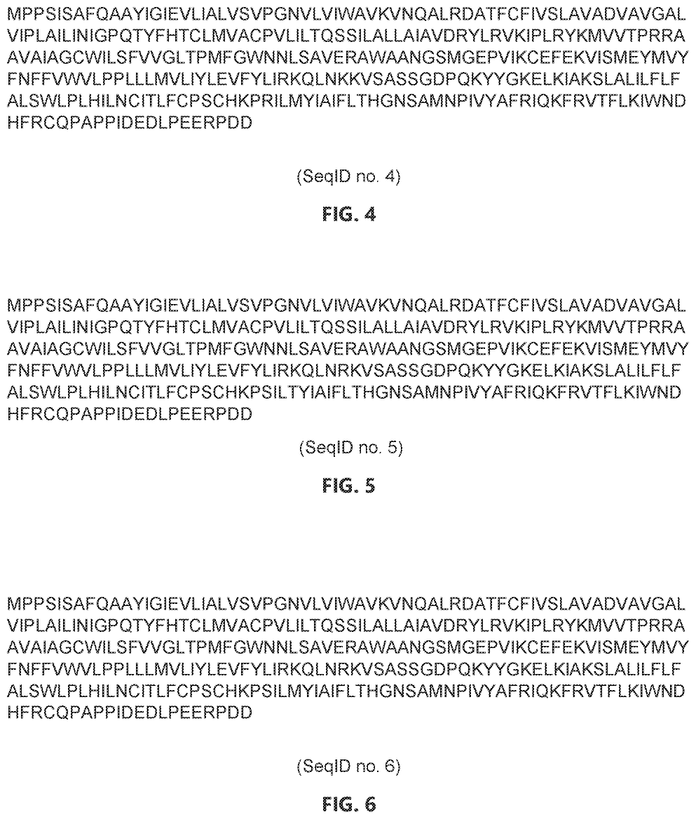

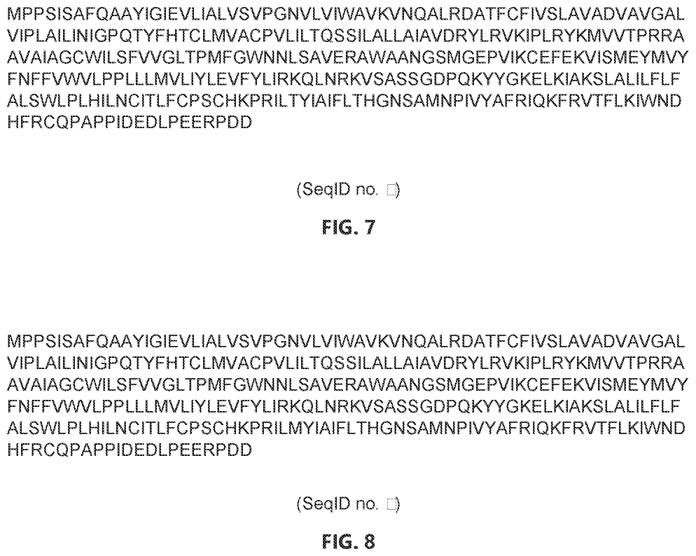

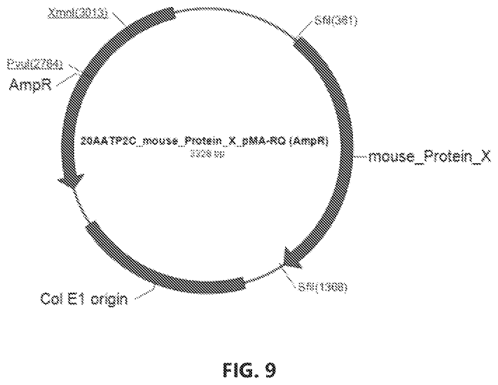

1. A composition for mutant amino acid sequences for the cDNA for the A1 adenosine receptor for inclusion in a plasmid selected from the group comprising:
a. an amino acid sequence as set forth in FIG. 1 (SeqID no. 1);
b. an amino acid sequence as set forth in FIG. 2 (SeqID no. 2);
c. an amino acid sequence as set forth in FIG. 3 (SeqID no. 3);
d. an amino acid sequence as set forth in FIG. 4 (SeqID no. 4);
e. an amino acid sequence as set forth in FIG. 5 (SeqID no. 5);
f. an amino acid sequence as set forth in FIG. 6 (SeqID no. 6);
g. an amino acid sequence as set forth in FIG. 7 (SeqID no. 7); and
h. an amino acid sequence as set forth in FIG. 8 (SeqID no. 8).

2. The composition according to claim 1, wherein the composition is to be administered to an antigen presenting cell that is either ex vivo or in vivo.

3. The composition according to claim 2, wherein the antigen presenting cells are selected from the group consisting of monocytes, macrophages, dendritic cells, Langerhans cells, lymphocytes, hematopoietic stem cells, peripheral blood stem cells, peripheral blood mononuclear cells, and B cells.

4. The composition according to claim 1, which further comprises at least one composition selected from the group consisting of a co-inhibitory and a co-stimulatory cancer immunotherapy agent.

5. The composition according to claim 1, which further comprises a co-inhibitory agent.

6. The composition according to claim 5, wherein the co-inhibitory agent is an antibody against CTLA-4 and PD-1.

7. The composition according to claim 1, which comprises co-stimulatory agent selected from the group consisting of GM-CSF, cytokines including interferons (IFNs), including INF-α, INF-β, and INF-γ, toll receptor agonists, IL-2, IL-15, IL-17, IL-21, and IL-7, tumor specific antigens, tumor associated antigens, and tumor neoantigens.

8. The composition according to claim 1, which further comprises at least one composition selected from the group consisting of a priming agent, A1 adenosine receptor agonists, A2a adenosine receptor antagonists, adenosine deaminase, protein kinase inhibitors, tyrosine phosphatase inhibitors, allosteric enhancers and agents which increase the expression of A1 adenosine receptors.

9. The composition according to claim 1, which is formulated for administration as an inhalational, intravenous, subcutaneous, topical, intratumor, rectal, or an organ in which the tumor is expressed.

10. The composition according to claim 1, which is formulated for administration as a lipid nanoparticle, with an oncolytic virus, as a patch with or without microneedles for subcutaneous or topical delivery, implants, scaffold nanoparticles, scaffolds, including biomaterial implant scaffolds, injectable biomaterial scaffolds, biomaterials, cell-based platforms, controlled release systems, and transdermal delivery systems.

11. The composition according to claim 10, which comprises a lipid nanoparticle consisting of DOTAP, DSPC, cholesterol, and DSPE-PEG2000-maleimide.

12. A method of inhibiting the proliferation of a cancer cell and reducing the size of a tumor comprising multiple cancer cells, the method comprising:
a) administering a plasmid directly to the cancer cell, tumor, or an organ in which the tumor is expressed a mutant A1 adenosine receptor plasmid cDNA wherein the plasmid delivers one or more mutant A1 adenosine receptors selected from the group consisting of:
i. an amino acid sequence as set forth in FIG. 1 (SeqID no. 1):
ii. an amino acid sequence as set forth in FIG. 2 (SeqID no. 2);
iii. an amino acid sequence as set forth in FIG. 3 (SeqID no. 3);
iv. an amino acid sequence as set forth in FIG. 4 (SeqID no. 4);
v. an amino acid sequence as set forth in FIG. 5 (SeqID no. 5);
vi. an amino acid sequence as set forth in FIG. 6 (SeqID no. 6);
vii. an amino acid sequence as set forth in FIG. 7 (SeqID no. 7); and
viii. an amino acid sequence as set forth in FIG. 8 (SeqID no. 8).

13. The method according to claim 12, which further comprises at least one composition selected from the group consisting of a co-inhibitory and a co-stimulatory cancer immunotherapy agent.

14. The method according to claim 13, which comprises co-inhibitory agents, such as an antibody against CTLA-4 and PD-1.

15. The method according to claim 13, which comprises co-stimulatory agents such as GM-CSF, cytokines including interferons (IFNs), including INF-α, INF-β, and IFN-γ, toll receptor agonists, IL-2, IL-15, IL-17, IL-21, and IL-7, tumor specific antigens, tumor associated antigens, tumor neoantigens.

16. The method according to claim 12, which further comprises at least one composition selected from the group consisting of a priming agent, A1 adenosine receptor agonists, A2a adenosine receptor antagonists, adenosine deaminase, protein kinase inhibitors, tyrosine phosphatase inhibitors, allosteric enhancers and agents which increase the expression of A1 adenosine receptors administered in combination with one of the amino acid sequences set forth in claim 12.

17. The method according to claim 12, which further comprises administering the composition of a mutant plasmid cDNA for the A1 adenosine receptor comprising one of the amino acid sequences set forth in claim 12 as an inhalational, intravenous, subcutaneous, topical, intratumor, rectal, or an organ in which the tumor is expressed.

18. The method of claim 12, which further comprises administering the composition of a mutant plasmid cDNA for the A1 adenosine receptor comprising one of the amino acid sequences set forth in claim 12 formulated as a lipid nanoparticle, with an oncolytic virus, as a patch with or without microneedles for subcutaneous or topical delivery, implants, scaffold nanoparticles, scaffolds including biomaterial implant scaffolds and injectable biomaterial scaffolds, biomaterials, cell-based platforms, controlled release systems, and transdermal delivery.

19. The method according to claim 18, which comprises a lipid nanoparticle consisting of DOTAP, DSPC, cholesterol and DSPE-PEG2000-maleimide.

20. The method according to claim 12, which further comprises administering the composition of a mutant plasmid cDNA for the A1 adenosine comprising one of the amino acid sequences set forth in claim 12 as an adjuvant therapy combined with chemotherapy, radiation therapy, surgery, immunotherapies such as toll receptor agonists and oncolytic viruses, androgen deprivation therapy and ablative therapies, including high-intensity focused ultrasound, interstitial laser ablation therapy, and vascular targeted photodynamic therapy and cryotherapy.


SEQUENCE LISTING (8 sequences)

SEQ ID NO: 1 - protein, 326 aa, Artificial Sequence
  Synthetic Mutant A1 Adenosine Receptor Plasmid cDNAs
MPPYISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKTVVTQRRAAVAIAGCWILSLVVGLTPMFGWNNLSEVEQAWIANGSVGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPTCQKPRILIYIAIFLTHGNSAMNPIVYAFRIHKFRVTFLKIWNDHFRCQPKPPIEEDIPEEKADD

SEQ ID NO: 2 - protein, 326 aa, Artificial Sequence
  Synthetic Mutant A1 Adenosine Receptor Plasmid cDNAs
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPSCHKPSILMYIAIFLTHGNSAMNPIVYAFRIQKFRVTFLKIWNDHFRCQPAPPIDEDLPEERPDD

SEQ ID NO: 3 - protein, 326 aa, Artificial Sequence
  Synthetic Mutant A1 Adenosine Receptor Plasmid cDNAs
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPSCHKPRILTYIAIFLTHGNSAMNPIVYAFRIQKFRVTFLKIWNDHFRCQPAPPIDEDLPEERPDD

SEQ ID NO: 4 - protein, 326 aa, Artificial Sequence
  Synthetic Mutant A1 Adenosine Receptor Plasmid cDNAs
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPSCHKPRILMYIAIFLTHGNSAMNPIVYAFRIQKFRVTFLKIWNDHFRCQPAPPIDEDLPEERPDD

SEQ ID NO: 5 - protein, 326 aa, Artificial Sequence
  Synthetic Mutant A1 Adenosine Receptor Plasmid cDNAs
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNRKVSASSGDPQKYYGKELKIAKSLALILFLFALSWLPLHILNCITLFCPSCHKPSILTYIAIFLTHGNSAMNPIVYAFRIQKFRVTFLKIWNDHFRCQPAPPIDEDLPEERPDD

SEQ ID NO: 6 - protein, 326 aa, Artificial Sequence
  Synthetic Mutant A1 Adenosine Receptor Plasmid cDNAs
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNRKVSASSGDPQKYYGKELKIAKSLALILFLFALSWLPLHILNCITLFCPSCHKPSILMYIAIFLTHGNSAMNPIVYAFRIQKFRVTFLKIWNDHFRCQPAPPIDEDLPEERPDD

SEQ ID NO: 7 - protein, 326 aa, Artificial Sequence
  Synthetic Mutant A1 Adenosine Receptor Plasmid cDNAs
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNRKVSASSGDPQKYYGKELKIAKSLALILFLFALSWLPLHILNCITLFCPSCHKPRILTYIAIFLTHGNSAMNPIVYAFRIQKFRVTFLKIWNDHFRCQPAPPIDEDLPEERPDD

SEQ ID NO: 8 - protein, 326 aa, Artificial Sequence
  Synthetic Mutant A1 Adenosine Receptor Plasmid cDNAs
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNRKVSASSGDPQKYYGKELKIAKSLALILFLFALSWLPLHILNCITLFCPSCHKPRILMYIAIFLTHGNSAMNPIVYAFRIQKFRVTFLKIWNDHFRCQPAPPIDEDLPEERPDD